(19) United States
(12) Patent Application Publication
(10) Pub. No.: US 2022/0202478 A1
(43) Pub. Date: Jun. 30, 2022

(54) ELECTROSURGICAL VESSEL SEALER HAVING OPPOSED SEALING SURFACES WITH VARYING GAP HEIGHT

(71) Applicant: Conmed Corporation, Utica, NY (US)

(72) Inventors: Derek Eilers, Denver, CO (US); Mason Williams, Centennial, CO (US)

(73) Assignees: Conmed Corporation, Utica, NY (US); Conmed Corporation, Utica, NY (US)

(21) Appl. No.: 17/607,560

(22) PCT Filed: Apr. 29, 2020
(86) PCT No.: PCT/US2020/030551

§ 371 Date: Oct. 29, 2021

Related U.S. Application Data
(60) Provisional application No. 62/840,437, filed on Apr. 30, 2019.

(51) Int. Cl. A61B 18/14

(52) U.S. Cl. CPC A61B 18/1482; A61B 2018/0063; A61B 18/1445

(57) ABSTRACT

An electrosurgical instrument is disclosed which includes a proximal handle portion, an elongated tubular body portion extending distally from the proximal handle portion, and a jaw assembly operatively associated with a distal end of the body portion and including a pair of cooperating jaw members mounted for movement between an open position and a closed position, each jaw member having a sealing surface, wherein the sealing surfaces of the jaw members define a vessel sealing gap therebetween when the jaw members are in the closed position, and wherein the vessel sealing gap has a height that varies along an axial extent of the jaw assembly between a proximal end portion of the jaw assembly and a distal end portion of the jaw assembly.

### CROSS-REFERENCE TO RELATED APPLICATION

This application is a U.S. National Phase Application filed under 35 U.S.C. § 371, based on International Patent Application No. PCT/US202/030551, filed Apr. 29, 2020, which claims priority to U.S. Provisional Patent Application Ser. No. 62/840,437 filed Apr. 30, 2019, the disclosures of which are herein incorporated by reference in their entirety.

### BACKGROUND OF THE INVENTION

### 1. Field of the Invention

The subject invention is directed to electrosurgical instruments, and more particularly, to a bi-polar vessel sealer having a jaw assembly that has opposed sealing surfaces with a varying tissue gap height.

### 2. Description of Related Art

Laparoscopic or “minimally invasive” surgical techniques are becoming commonplace in the performance of procedures such as cholecystectomies, appendectomies, hernia repair and nephrectomies. Benefits of such procedures include reduced trauma to the patient, reduced opportunity for infection, and decreased recovery time. Such procedures within the abdominal (peritoneal) cavity are typically performed through a device known as a trocar or cannula, which facilitates the introduction of laparoscopic instruments into the abdominal cavity of a patient.

Electrosurgical instruments for sealing blood vessels are often used in laparoscopic and other endoscopic surgical procedures. These instruments utilize both the mechanical clamping action of a pair of jaws and electrical energy to cauterize and seal blood vessels during a surgical procedure. Existing vessel sealing devices use non-conductive stops to create a gap between the sealing surfaces (electrodes) of the jaws without allowing current to transfer through the stops. This gap allows for energy to transfer through tissue, between the sealing surfaces (one side acting as the anode and the other as the cathode) and is a critical feature in providing effective sealing. The prior art describes the stops added to opposing sealing surfaces as being designed with a uniform gap between the surfaces. An example of such a prior art device is disclosed in U.S. Pat. No. 10,568,682.

In addition to controlling the gap between electrodes, tissue grasping is also a crucial aspect of jaw design, especially when dividing tissue. In bi-polar sealers, tissue is typically divided with a cutting blade that runs through the center of the jaws that creates an axial force on the tissue when deployed. If there isn't sufficient grasping of the tissue, the tissue will be forced out of the jaws during use. It would be beneficial therefore to provide an electrosurgical vessel sealing instrument that uses non-conductive stops on opposing sealing surfaces to provide gap control but also includes a non-uniform separation between the sealing surfaces to aid in tissue grasping.

### SUMMARY OF THE DISCLOSURE

The subject invention is directed to a new and useful electrosurgical instrument for use in endoscopic and laparoscopic surgical procedures to cauterize and seal blood vessels using electrical energy, which has enhanced tissue grasping characteristics. The electrosurgical instrument includes a proximal handle portion, an elongated tubular body portion that extends distally from the proximal handle portion and a jaw assembly that is operatively associated with a distal end of the tubular body portion.

The jaw assembly includes a pair of cooperating jaw members that are adapted and configured for movement between an open position and a closed position. Each jaw member includes a conductive sealing plate upon which a sealing surface of the jaw member is defined. The two sealing surfaces of the jaw members define a vessel sealing gap therebetween when the jaw members are in the closed position. Preferably, the vessel sealing gap has a height that varies along an axial extent of the jaw assembly between a proximal end portion of the jaw assembly and a distal end portion of the jaw assembly. This varying height vessel sealing gap enhances the tissue grasping characteristics of the jaw assembly.

More particularly, the vessel sealing gap of the jaw assembly includes a proximal gap area, a medial gap area and a distal gap area. The height of the medial gap area is greater than the height of the proximal gap area and the height of the distal gap area. It is envisioned that at least one of the jaw members includes a proximal sealing surface, a medial sealing surface and a distal sealing surface, and the height of the medial sealing surface is less than the height of the proximal sealing surface and the height of the distal sealing surface.

At least a portion of the sealing surface of each jaw member has a plurality of spaced apart coining features formed therein for enhancing the tissue grasping characteristics of the jaw assembly. In addition, at least a portion of the sealing surface of each jaw member has a plurality of spaced apart non-conductive protuberances formed thereon for grasping tissue. The protuberances act as stops to help define the vessel sealing gap and to further enhance the tissue grasping characteristics of the jaw assembly.

Preferably, the non-conductive protuberances are formed on the sealing surface of each jaw member from a ceramic material in an additive manufacturing process, and they are preferably located in the proximal gap area, the medial gap area and the distal gap area. It is envisioned that the location, spacing, size and shape of non-conductive protuberances or stops could vary by design to enhance or otherwise change the tissue grasping characteristics of the jaw assembly.

A conductive wire extends from the proximal handle assembly, through the elongated body to the jaw assembly for connecting with each of the conductive sealing plates to supply energy thereto for sealing a blood vessel. The sealing surface on each jaw member includes a recessed track for accommodating a translating cutting blade that is used to divide a sealed blood vessel. The proximal handle portion includes a deployment trigger operatively connected to the jaw assembly through the elongated body portion for moving the cutting blade through the jaw assembly within the recessed track formed in in each sealing surface.

The proximal handle portion further includes an actuation handle operatively connected to the jaw assembly through the elongated body portion for moving the jaw members between the open and closed positons. The proximal handle portion also includes a rotation knob operatively associated with the elongated body portion for rotating the elongated body portion about a longitudinal axis thereof relative to the proximal handle portion.

Each jaw member includes a proximal yoke portion having an angled cam slot formed therein for accommodating a transverse cam pin that is operatively connected to the actuation handle through the elongated body portion, and an aperture for accommodating a transverse pivot pin.

The subject invention is also directed to an electrosurgical instrument for use in endoscopic and laparoscopic surgical procedure to seal and divide a blood vessel, which includes a proximal handle portion, an elongated tubular body portion extending distally from the proximal handle portion, a jaw assembly operatively associated with a distal end of the body portion and including a pair of cooperating jaw members mounted for movement between an open position and a closed position for grasping and sealing a blood vessel, and a cutting blade operatively associated with the jaw assembly for dividing the sealed blood vessel.

Preferably, each jaw member of the jaw assembly includes a conductive sealing plate upon which a sealing surface of the jaw member is defined, and the opposed sealing surfaces of the jaw members define a vessel sealing gap therebetween when the jaw members are in the closed position. The vessel sealing gap includes a proximal gap area, a medial gap area and a distal gap area, wherein the height of the medial gap area is greater than the height of the proximal gap area and the height of the distal gap area so as to provide the jaw assembly with enhanced tissue grasping characteristics, particularly when the sealed blood vessel is being divided by the cutting blade.

These and other features of the electrosurgical instrument of the subject invention will become more readily apparent to those having ordinary skill in the art to which the subject invention appertains from the detailed description of the preferred embodiments taken in conjunction with the following brief description of the drawings.

### BRIEF DESCRIPTION OF THE DRAWINGS

So that those skilled in the art will readily understand how to make and use the electrosurgical instrument of the subject invention without undue experimentation, preferred embodiments thereof will be described in detail herein below with reference to the figures wherein:

FIG. 1 is a perspective view of the electrosurgical instrument of the subject invention with the jaw assembly in a closed position grasping a blood vessel;

FIG. 2 is an enlarged localized view of the jaw assembly as shown in FIG. 1;

FIG. 3 is a side elevation view of the jaw assembly in a closed position illustrating the vessel sealing gap;

FIG. 4 is an enlarged localized view of the distal portion of the jaw assembly as shown in FIG. 3;

FIG. 5 is an enlarged localized view of the medial portion of the jaw assembly, illustrating the non-conductive stops on the opposed sealing surfaces as shown in FIG. 3;

FIG. 6 is an enlarged localized view of the proximal portion of the jaw assembly as shown in FIG. 3;

FIG. 7 is a perspective view of the jaw assembly in an open position;

FIG. 8 is a perspective view of the upper jaw of the jaw assembly, separated from the instrument;

FIG. 9 is a an exploded perspective view of the upper jaw member shown in FIG. 8, with parts separated for ease of illustration;

FIG. 10 is a side elevation of the handle assembly of the electrosurgical instrument of the subject invention, in cross-section taken along line 10-10 of FIG. 1, showing the stroke of the actuation handle used to move the jaw assembly between its open and closed positions;

FIG. 11 is a side elevation of the jaw assembly showing the movement of the jaws between their open and closed positions;

FIG. 12 is a side elevation of the handle assembly of the electrosurgical instrument of the subject invention, in cross-section taken along line 10-10 of FIG. 1, showing the stroke of the deployment trigger used to actuate the cutting knife; and

FIG. 13 is a local perspective view of the closed jaw assembly, with upper jaw member separated from the lower jaw member so as to reveal the travel of the cutting knife.

### DETAILED DESCRIPTION OF THE PREFERRED EMBODIMENTS

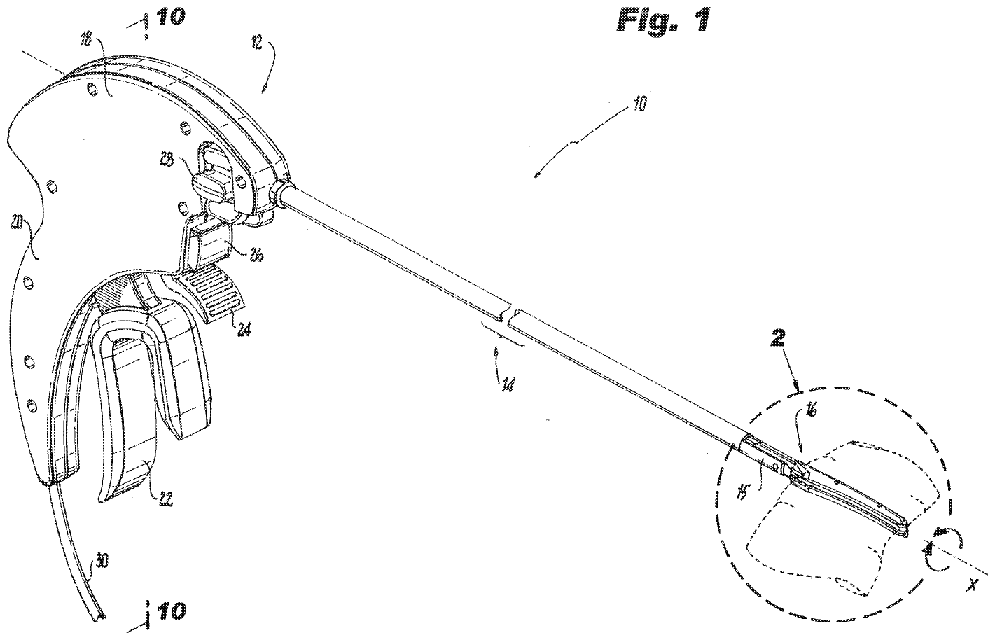

Referring now to the drawings wherein like reference numerals identify like or similar structural elements or features of the subject invention, there is illustrated in FIG. 1 an electrosurgical instrument, which is constructed in accordance with a preferred embodiment of the subject invention and designated generally by reference numeral 10. The electrosurgical instrument 10 is adapted and configured for use in endoscopic and laparoscopic surgical procedures to cauterize and seal blood vessels using electrical energy. and to subsequently divide the sealed and cauterizing blood vessel. The instrument 10 is preferably sized for use with a 5 mm access port or trocar. However, it can be scaled up for use with larger access ports.

The electrosurgical instrument 10 of the subject invention includes a proximal handle assembly 12, an elongated tubular body portion 14 that extends distally from the proximal handle assembly 12 and a bi-polar jaw assembly 16 that is operatively associated with a distal end of the tubular body portion 14. More particularly, the tubular body portion 14 includes a bifurcated distal end section 15 that accommodates the bi-polar jaw assembly 16.

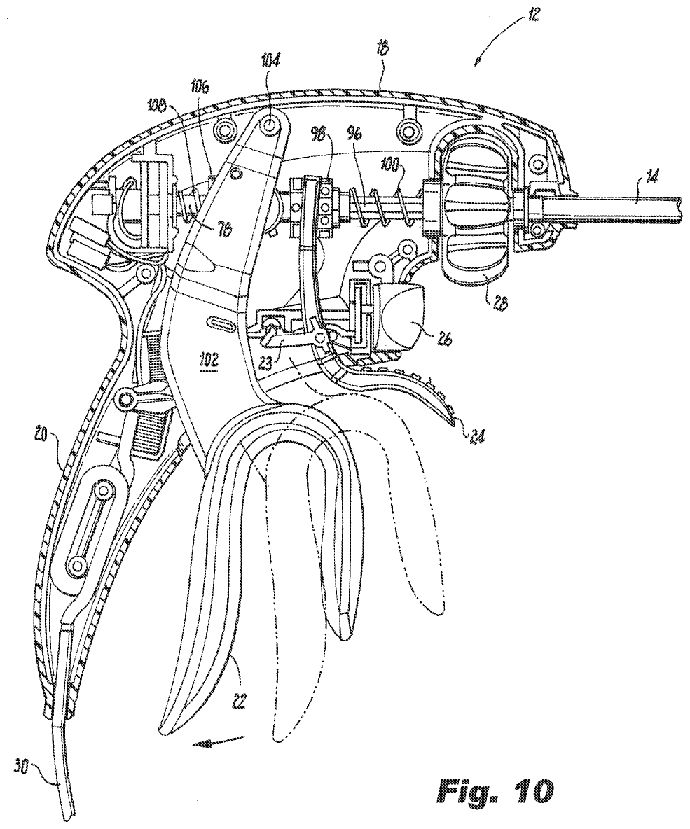

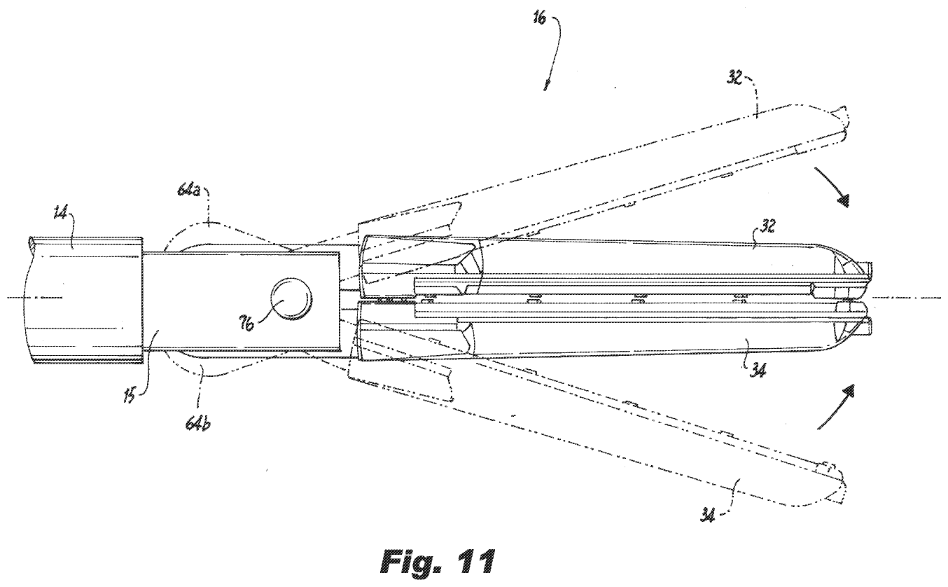

The proximal handle assembly 12 is preferably formed in two-parts from a high strength, light weight medical grade plastic material, such as Lexan or the like, and it includes an upper body portion 18 and a lower fixed grasping portion 20. A U-shaped pivoting actuation handle 22 is operatively associated with the upper body portion 18 of the handle assembly 12 for actuating the jaw assembly 16, as will be discussed in more detail below with further reference to FIGS. 10 and 11.

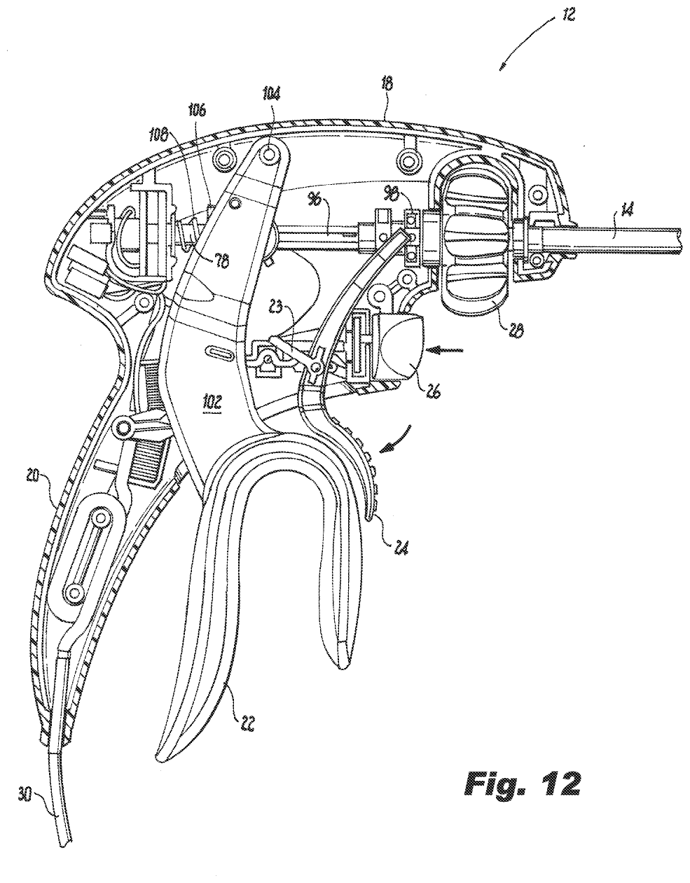

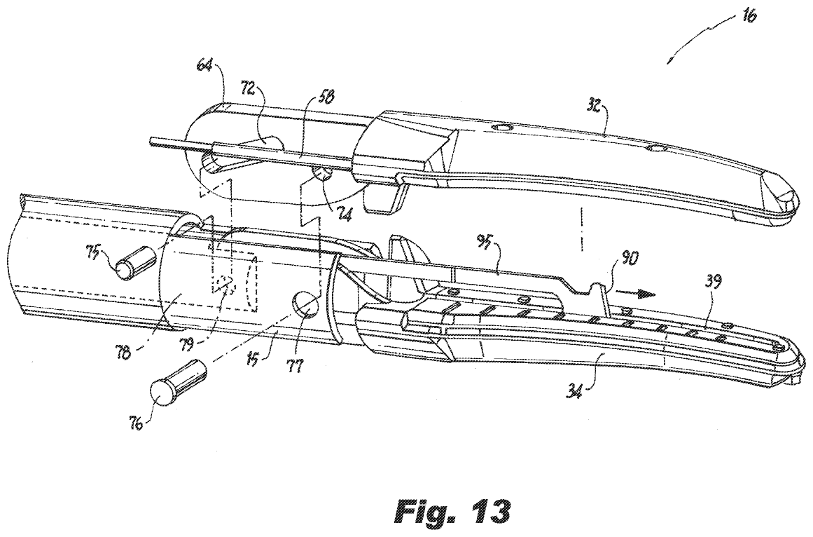

A deployment trigger 24 is also operatively associated with the body portion 18 of the handle assembly 12 for actuating a cutting knife that translates through the jaw assembly 16 to divide a sealed blood vessel, which will also be discussed in more detail below with further reference to FIGS. 12 and 13. A trigger lock 26 is operatively associated with the trigger 24 to prevent unintended actuation of the knife during use.

With continuing reference to FIG. 1, a rotation knob 28 is operatively associated with the body portion 18 of handle assembly 12 for rotating the tubular body portion 14 and the jaw assembly 16 about the longitudinal axis X of the tubular body portion 14 relative to the handle assembly 12. A power cable 30 extends from the fixed grasping portion 20 of handle assembly 12 to connect the instrument 10 to an energy source.

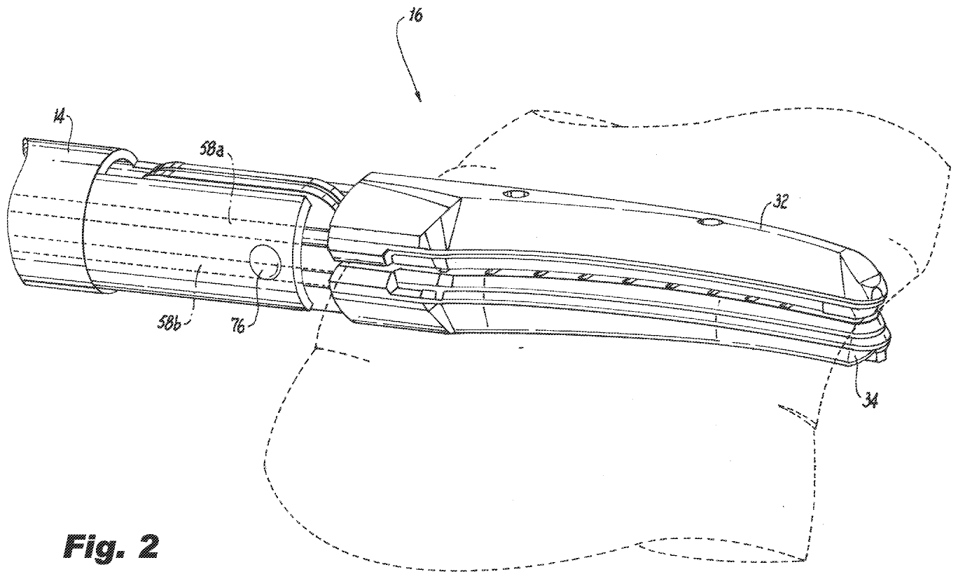

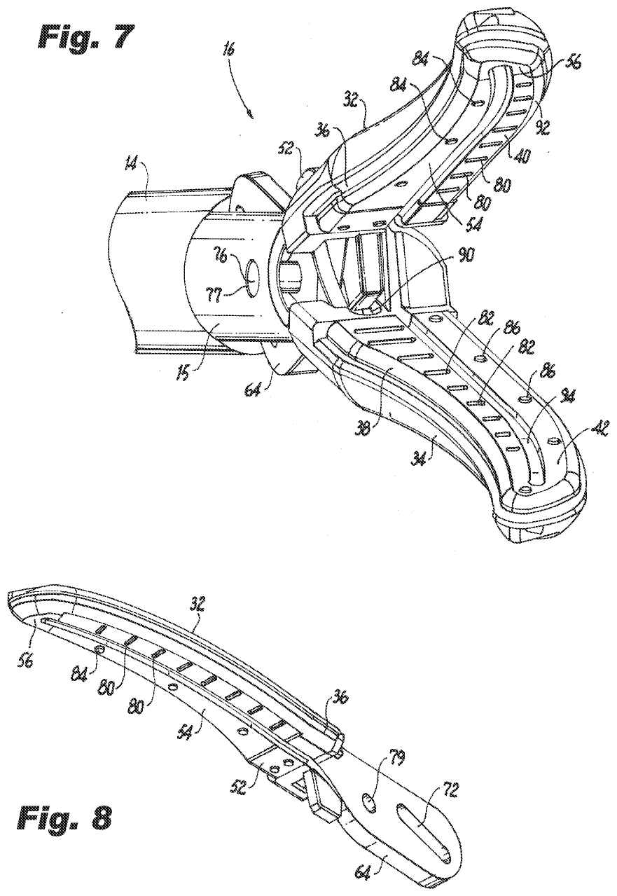

Referring now to FIGS. 2 through 9, the bi-polar jaw assembly 16 of electrosurgical instrument 10 includes a pair of cooperating jaw members 32 and 34, where jaw member 32 is the upper jaw of the assembly 16 and jaw member 34 is the lower jaw of the assembly 16. The jaw assembly 16 is adapted and configured for controlled movement between a closed position shown for example in FIG. 2 and an open position shown for example in FIG. 7, which is accomplished through the manual movement of the actuation handle 22 relative to the fixed grasping portion 20 of handle assembly 12, as discussed in more detail below.

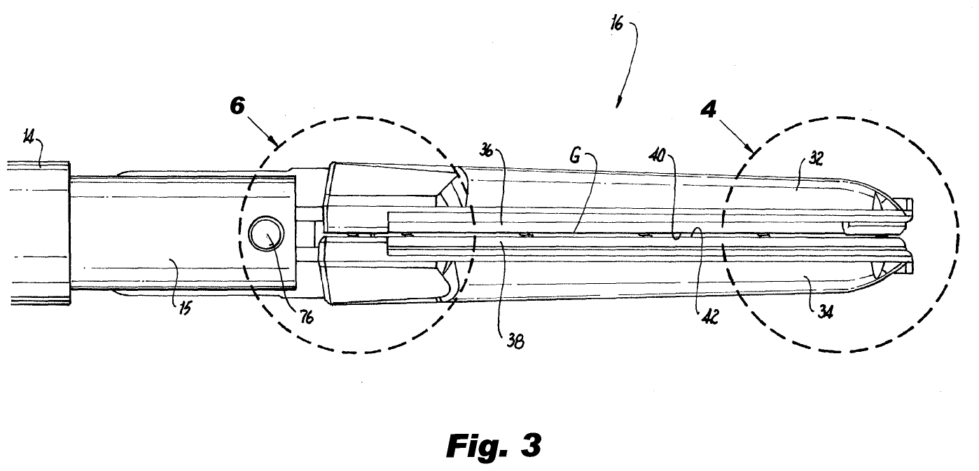

As best seen in FIG. 7, each jaw member 32, 34 of jaw assembly 16 includes a conductive seal plate 36, 38 upon which a sealing surface 40, 42 of the jaw member is defined. The two sealing surfaces 40, 42 of the jaw members 32, 34 define a vessel sealing gap G therebetween when the jaw members 32, 34 are in the closed position, as best illustrated in FIG. 3. Preferably, the vessel sealing gap G has a height that varies along an axial extent of the jaw assembly 16 between a proximal end portion of the jaw assembly 16 and a distal end portion of the jaw assembly 16, within a range of between 0.001 inches and 0.006 inches. This serves to advantageously enhance the tissue grasping characteristics of the jaw assembly 16 so that tissue is not forced out of the jaw assembly when the sealed vessel is divided.

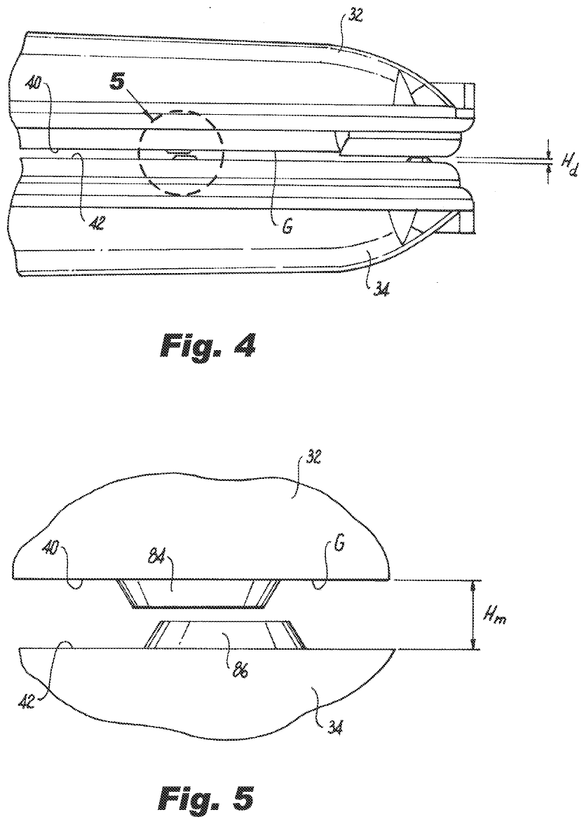

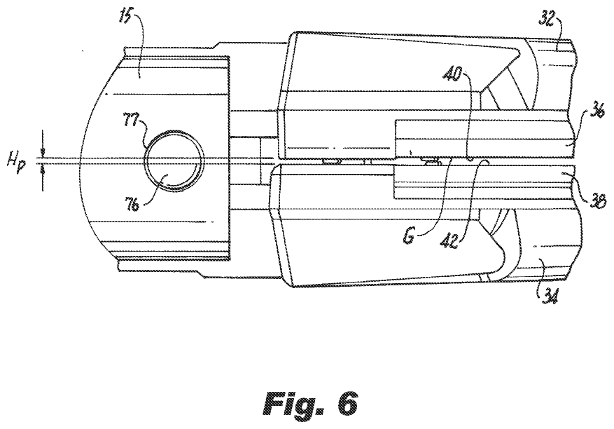

The vessel sealing gap G of the jaw assembly 16 includes a distal gap area that is best seen in FIG. 4, a medial gap area that is best seen in FIG. 5 and a proximal gap area that is best seen in FIG. 6. In accordance with a preferred embodiment of the subject invention, the height Hₘ of the medial gap area shown in FIG. 5 is greater than the height H_{d} of the distal gap area shown in FIG. 4 and the height Hₚ of the proximal gap area shown in FIG. 6. In order to accomplish this varying gap height, it is envisioned that at least one of the jaw members 32, 34 includes a proximal sealing surface, a medial sealing surface and a distal sealing surface, wherein the height of the medial sealing surface is less than the height of the proximal sealing surface and the height of the distal sealing surface.

By way of illustrative example, as best seen in FIGS. 7 and 8, the sealing surface 40 of the sealing plate 36 of the upper jaw member 32 includes a proximal sealing surface 52, a medial sealing surface 54 and a distal sealing surface 56, wherein and the height of the medial sealing surface 54 is less than the height of the proximal sealing surface 52 and the height of the distal sealing surface 56.

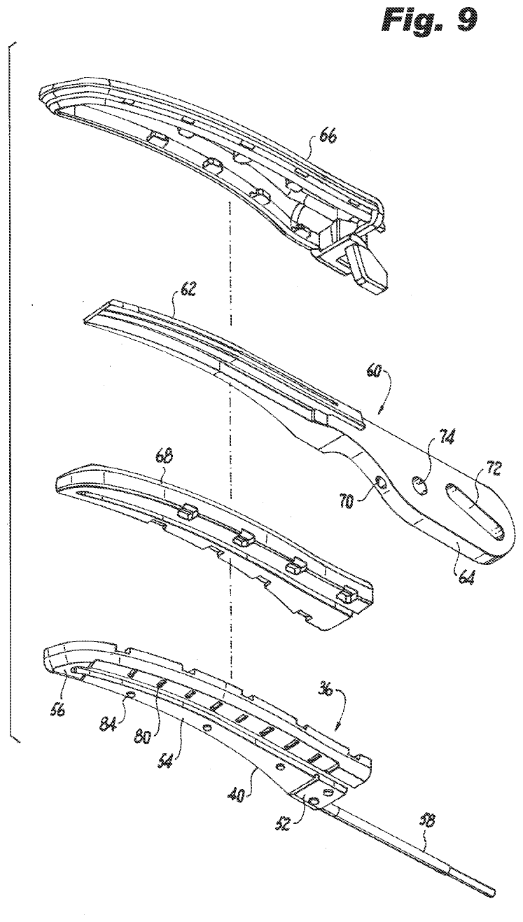

Referring now to FIGS. 8 and 9, in addition to the conductive sealing plate 36, the upper jaw member 32 of jaw assembly 16 includes a main jaw body 60 that includes a distal beam portion 62 and a proximal yoke portion 64. The distal beam portion 62 is sandwiched between upper and lower cover members 66 and 68, that are made from an injection molded plastic material. The upper sealing plate 36 is secured to the upper cover member 68, so that the conductive sealing plate 36 is insulated from the main jaw body 60. In addition, the upper sealing plate 36 is attached by welding to an electrical conductor 58 that carries electrical energy from the handle assembly 12, through the elongated body portion 14 to the upper jaw 32 of jaw assembly 16 for sealing a blood vessel.

The proximal yoke portion 64 of jaw member 32 has a longitudinal bore hole 70 for accommodating passage of the electrical conductor 58, an angled cam slot 72 for accommodating a transverse camming pin 75 (see FIG. 13) that is operatively connected to the actuation handle 22 through the elongated body portion 14, and an aperture 74 for accommodating a transverse pivot pin 76 which is supported in port 77 in the bifurcated distal section 15 of body portion 14. (See FIG. 13). The camming pin 75 is secured in an aperture 79 in the distal end of the actuation shaft 78 that extends through the elongated body portion 14 to the proximal handle assembly 12, and is operatively associated with the actuation handle 22, as discussed in more detail below.

Those having ordinary skill in the art will readily appreciate that the structure of the lower jaw member 34 of jaw assembly 16 is substantially similar to the structure of the upper jaw member 32 of jaw assembly 16 described above, except that the angled cam slot in the proximal yoke of the lower jaw member 34 would be oppositely oriented so that longitudinal movement of the camming pin 75 relative to the two oppositely angled cam slots would effectuate the opening and closing of the two jaw members 32, 34. Also, note the paired conductors 58a, 58b in shown FIG. 2 and the paired yoke portions 64a, 64b shown in FIG. 11.

More particularly, with reference to FIGS. 10 and 11, in use, manual approximation of the actuation handle 22 towards the fixed handle portion 20 of handle assembly 12 causes the integral rocker arm 102 of actuation handle 22 to pivot about the pin 104 in the body portion 18. This motion causes the coupling 106 to move in a distal direction, which drives the actuation shaft 78 in a distal direction within the tubular body portion 14. This advances the camming pin 75 is a distal direction with respect to the angled cam slots (e.g., cam slot 72) in the proximal yoke portions of each jaw member 32, 34. As a result, the two jaw members 32, 34 approximate toward one another into a closed positon.

Once closed, the bi-polar jaw assembly 16 is energized to seal and cauterize a blood vessel grasped between the conductive sealing surfaces 40, 42. Those skilled in the art will readily appreciate that the control of electrical power to the instrument 10 by way of power cable 30 can be achieved through actuation of a foot peddle or other mechanism connected to the power cable 30. Thereafter, upon the release of actuation handle 22, the actuation shaft 78 will be pulled in a proximal direction under the influence of the coiled spring 108 associated with the coupling 106 of the rocker arm 102.

Referring again to FIGS. 8 and 9, in conjunction with FIG. 7, at least a portion of the sealing surface 40, 42 of each jaw member 32, 34 has a plurality of spaced apart coining features formed therein to enhance the tissue grasping characteristics of the jaw assembly 16. More particularly, a section of the sealing surface 40 of the upper jaw member 32 includes a set of spaced apart rectangular coining features 80, while a mirrored section of the sealing surface 42 of the lower jaw member 34 includes a corresponding set of spaced apart rectangular coining features 82.

In addition, at least a portion of the sealing surface 40, 42 of each jaw member 32, 34 has a plurality of spaced apart non-conductive protuberances formed thereon for further enhancing the tissue grasping characteristics of the jaw assembly 16. More particularly, a section of the sealing surface 40 of the upper jaw member 32 includes a set of spaced apart rounded protuberances 84, while a mirrored section of the sealing surface 42 of the lower jaw member 34 includes a corresponding set of spaced apart rounded protuberances 86. The protuberances also act as stops to maintain the gap spacing between the conductive sealing surfaces 40, 42 of the jaw members 32, 34.

The geometry of the non-conductive protuberances 84, 86 is best seen in FIG. 5. Preferably, the non-conductive protuberances 84, 86 are formed on the sealing surfaces 40, 42 of each jaw member 32, 34 from a ceramic material in an additive manufacturing process. In a preferred embodiment of the subject invention, the manufacturing process involves high velocity oxy-fuel (HVOF) deposition. In this process, the sealing surfaces 40, 42 of the conductive sealing plates 36, 38 are cleaned and grit blasted to add surface roughness for better adhesion. The sealing plates 36, 38 are then loaded into a fixture and a mask is added that has opening to define the location of each protuberance 84, 86. The ceramic material is then sprayed on to the masked surfaces in layers at a high velocity and temperature until the appropriate height is achieved.

The protuberances 84, 86 are preferably, but not necessarily located in the proximal gap area, the medial gap area and the distal gap area defined between the two jaw members 32, 34. It is envisioned that the location, spacing, size and shape of non-conductive protuberances 84, 86 could vary by design to enhance or otherwise change the tissue grasping characteristics of the jaw assembly.

Referring now to FIGS. 12 and 13, in conjunction with FIG. 6, the opposed sealing surfaces 40, 42 on the two jaw members 32, 34 of jaw assembly 16 include recessed tracks 92, 94 for accommodating a translating cutting blade 90 that is used to divide a sealed blood vessel. In this regard, the deployment trigger 24 is operatively connected to the cutting blade 90 by way of a drive shaft 96 that extends from a trigger coupling 98, through the tubular body portion 14 to the shank 95 of the cutting blade 90 within jaw assembly 16.

In use, upon pressing the trigger lock 26 to displace the pivoting lock link 23, manual actuation of the trigger 24 against the bias of the coiled spring 100 that surrounds the drive shaft 96, causes the drive shaft 96 to advance in a distal direction. This drives the cutting blade 90 through the jaw assembly 16 within the recessed tracks 92, 94 in jaw members 32, 34 to divide a sealed blood vessel, as best seen in FIG. 13. At such a time, the sealed blood vessel in firmly gripped between the jaw member 32, 34 of jaw assembly 16, held securely by opposed sets of spaced apart rectangular coining features 80, 82 and the opposed sets of spaced apart rounded protuberances 84, 86, as well as the varying height of the vessel sealing gap G defined between the opposed sealing surfaces 40, 42.

While the electrosurgical instrument of the subject disclosure has been shown and described with reference to preferred embodiments, those skilled in the art will readily appreciate that changes and/or modifications may be made thereto without departing from the scope of the subject disclosure.

What is claimed is:

1. An electrosurgical instrument, comprising:
a) a proximal handle portion;
b) an elongated tubular body portion extending distally from the proximal handle portion; and
c) a jaw assembly operatively associated with a distal end of the body portion and including a pair of cooperating jaw members that are adapted and configured for movement between an open position and a closed position, each jaw member having a sealing surface, wherein the sealing surfaces of the jaw members define a vessel sealing gap therebetween when the jaw members are in the closed position, and wherein the vessel sealing gap has a height that varies along an axial extent of the jaw assembly between a proximal end portion of the jaw assembly and a distal end portion of the jaw assembly.

2. An electrosurgical instrument as recited in claim 1, wherein the vessel sealing gap includes a proximal gap area, a medial gap area and a distal gap area, and wherein a height of the medial gap area is greater than a height of the proximal gap area and a height of the distal gap area.

3. An electrosurgical instrument as recited in claim 2, wherein at least one of the jaw members includes a proximal sealing surface, a medial sealing surface and a distal sealing surface, and wherein a height of the medial sealing surface is less than a height of the proximal sealing surface and a height of the distal sealing surface.

4. An electrosurgical instrument as recited in claim 3, wherein at least a portion of the sealing surface of each jaw member has a plurality of spaced apart coining features formed therein for grasping tissue.

5. An electrosurgical instrument as recited in claim 3, wherein at least a portion of the sealing surface of each jaw member has a plurality of spaced apart non-conductive protuberances formed thereon for grasping tissue.

6. An electrosurgical instrument as recited in claim 5, wherein the non-conductive protuberances are formed on the sealing surface of each jaw member from a ceramic material in an additive manufacturing process.

7. An electrosurgical instrument as recited in claim 5, wherein the non-conductive protuberances are formed in the proximal gap area, the medial gap area and the distal gap area.

8. An electrosurgical instrument as recited in claim 1, wherein the seal surface on each jaw member includes a recessed track for accommodating a blade for dividing a sealed vessel.

9. An electrosurgical instrument as recited in claim 1, wherein each jaw member includes a conductive sealing plate upon which the sealing surface of the jaw member is defined.

10. An electrosurgical instrument as recited in claim 1, wherein a conductive wire extends from the proximal handle assembly, through the elongated body to the jaw assembly for connecting with each of the conductive sealing plates.

11. An electrosurgical instrument as recited in claim 8, wherein the proximal handle portion includes an actuation trigger operatively connected to the jaw assembly through the elongated body portion for moving the cutting blade through the jaw assembly within the recessed track formed in in each sealing surface.

12. An electrosurgical instrument as recited in claim 1, wherein the proximal handle portion includes an actuation handle operatively connected to the jaw assembly through the elongated body portion for moving the jaw members between the open and closed positons.

13. An electrosurgical instrument as recited in claim 12, wherein each jaw member includes a proximal yoke portion having an angled cam slot formed therein for accommodating a transverse cam pin that is operatively connected to the actuation handle through the elongated body portion.

14. An electrosurgical instrument as recited in claim 12, wherein each jaw member includes a proximal yoke portion having an aperture formed therein for accommodating a transverse pivot pin.

15. An electrosurgical instrument as recited in claim 1, the proximal handle portion includes a rotation knob operatively associated with the elongated body portion for rotating the elongated body portion about a longitudinal axis thereof relative to the proximal handle portion.

16. An electrosurgical instrument for sealing and dividing a blood vessel, comprising:
a) a proximal handle portion;
b) an elongated tubular body portion extending distally from the proximal handle portion;
c) a bi-polar jaw assembly operatively associated with a distal end of the body portion and including a pair of cooperating jaw members mounted for movement between an open position and a closed position, each jaw member having a conductive sealing plate upon which a sealing surface of the jaw member is defined, wherein the sealing surfaces of the jaw members define a vessel sealing gap therebetween when the jaw members are in the closed position, wherein the vessel sealing gap includes a proximal gap area, a medial gap area and a distal gap area, and wherein a height of the medial gap area is greater than a height of the proximal gap area and a height of the distal gap area; and
d) a cutting blade operatively associated with the jaw assembly for moving through the sealing gap to divide a sealed vessel held within the sealing gap.

17. An electrosurgical instrument as recited in claim 16, wherein at least one of the jaw members includes a proximal sealing surface, a medial sealing surface and a distal sealing surface, and wherein a height of the medial sealing surface is less than a height of the proximal sealing surface and a height of the distal sealing surface.

18. An electrosurgical instrument as recited in claim 16, wherein at least a portion of the sealing surface of each jaw member has a plurality of spaced apart coining features formed therein for grasping tissue.

19. An electrosurgical instrument as recited in claim 16, wherein at least a portion of the sealing surface of each jaw member has a plurality of spaced apart non-conductive protuberances formed thereon for grasping tissue.

20. An electrosurgical instrument as recited in claim 19, wherein the non-conductive protuberances are formed on the sealing surface of each jaw member from a ceramic material in an additive manufacturing process.

21. An electrosurgical instrument as recited in claim 19, wherein the non-conductive protuberances are formed in the proximal gap area, the medial gap area and the distal gap area.

22. An electrosurgical instrument as recited in claim 16, wherein the seal surface on each jaw member includes a recessed track for accommodating movement of the cutting blade.

23. An electrosurgical instrument as recited in claim 16, wherein a conductive wire extends from the proximal handle assembly, through the elongated body to the jaw assembly for connecting with each of the conductive sealing plates.

24. An electrosurgical instrument as recited in claim 16, wherein the proximal handle portion includes an actuation trigger operatively connected to the jaw assembly through the elongated body portion for moving the cutting blade through the jaw assembly within the recessed track formed in in each sealing surface.

25. An electrosurgical instrument as recited in claim 16, wherein the proximal handle portion includes an actuation handle operatively connected to the jaw assembly through the elongated body portion for moving the jaw members between the open and closed positons.

26. An electrosurgical instrument as recited in claim 16, wherein each jaw member includes a proximal yoke portion having an angled cam slot formed therein for accommodating a transverse cam pin that is operatively connected to the actuation handle through the elongated body portion.

27. An electrosurgical instrument as recited in claim 26, wherein each jaw member includes a proximal yoke portion having an aperture formed therein for accommodating a transverse pivot pin.

28. An electrosurgical instrument as recited in claim 16, the proximal handle portion includes a rotation knob operatively associated with the elongated body portion for rotating the elongated body portion about a longitudinal axis thereof relative to the proximal handle portion.
